(19) United States
(12) Patent Application Publication
(10) Pub. No.: US 2014/0038241 A1
(43) Pub. Date: Feb. 6, 2014

(54) GENOMIC ENRICHMENT METHOD, COMPOSITION, AND REAGENT KIT

(71) Applicants: Zhaohui Zhou, San Ramon, CA (US); Qun Shan, Albany, CA (US)

(72) Inventors: Zhaohui Zhou, San Ramon, CA (US); Qun Shan, Albany, CA (US)

(21) Appl. No.: 13/958,553

(22) Filed: Aug. 3, 2013

Related U.S. Application Data
(60) Provisional application No. 61/679,725, filed on Aug. 5, 2012.

(51) Int. Cl. C12P 19/34; C12N 9/16

(52) U.S. Cl. CPC C12P 19/34; C12N 9/16
USPC 435/91.53; 435/196; 435/188; 435/174

(57) ABSTRACT

By using engineered sequence specific DNA nuclease (“SSDN”), the composition, reagent kit and method of the present invention can cut and release a DNA sequence of interest 1×10⁴-1×10⁷-base pairs long from a source DNA as large as the whole genome. The SSDN further includes an affinity tag or is bound to a solid support that facilitates the isolation of the DNA sequence of interest. The SSDN can include a RecA and Ref combination, a transcription activator like effector nuclease, or a sequence specific chemical nuclease. When applied to genomic sequencing, specific region(s) of interest in the genome can be cut and isolated. Because the irrelevant part of the genome is removed from the sequencing reaction, the speed, cost, and accuracy of genomic sequencing can be improved.

### CROSS REFERENCE TO RELATED APPLICATIONS

This application claims the benefit of U.S. Provisional application No. 61/679,725, filed Aug. 5, 2012, which is incorporated herein by reference in its entirety.

### FIELD OF THE DISCLOSURE

This invention relates to method, composition, and reagent kit for cutting out and isolating ds DNA fragment with sequence specificity from larger DNA piece or genomic DNA. An application of the invention is for genomic enrichment, for example, to isolate DNA fragments of diagnostic relevance from whole genome for DNA sequencing in clinical settings.

### BACKGROUND OF THE INVENTION

The advancement in next-generation sequencing technologies has improved our ability to sequence large genomes at a lower cost than ever before. However, whole genome sequencing is still time and cost prohibitive to be applied routinely in clinical settings. Contrary to common conception that a person only needs to have his/her gene sequenced once in a lifetime, sequencing may be required multiple times each for a specific purpose. For examples, in cancer diagnostics, heterogeneous cell populations such as tumor cells and normal cells would be sequenced at the same time. In analyzing disease progression, cells from the same source may need to be sequenced at different times. Sequencing may also be applied in prenatal diagnostics to specific cell populations.

In many applications, the goal is only to get an accurate picture of a certain region or regions of the genome of these particular cell populations. Without isolating the specific genomic region, whole genome sequencing is not only wasteful, but also causes delay and inaccuracy. Therefore, a genomic enrichment method that allows isolation of a specific region or regions of the genome will lower the cost of sequencing, improve accuracy, and cut time to result significantly.

A number of genomic enrichment methods have been reported. One method is PCR based, in which multiple PCR primers are designed and tested. However, PCR amplification and normalization process are labor intensive, and as a result, this method cannot be applied universally. In addition, PCR can only be used to amplify DNA fragments of certain limited size ranges, and complexity of the genome makes it hard to achieve high multiplex PCR with consistent result. A second method is based on sequence specific ligation followed by universal PCR. Again, ligation probe design, process optimization, and size limitation make it less than ideal. A third method is microarray hybridization based. A subset of genomic DNA sequences is captured based on complementary sequence identity. The captured DNA fragments are then broken down and go through the typical library construction protocol. The method can capture larger genomic DNA fragments, but it lacks specificity as it depends on hybridization and elution. The efficiency is low, cost is high, and it takes extra time for hybridization to work well.

### BRIEF SUMMARY OF THE INVENTION

Described herein are method, composition, and reagent kit for cleaving and purifying a DNA fragment of interest from a target DNA with sequence specificity, enabling genomic enrichment and selective genomic sequencing.

In an embodiment of the invention, a genomic enrichment method described herein employs a pair of engineered sequence specific DNA nucleases that bind a target DNA at a pair of binding sites that enclose a DNA fragment of interest, forming a pair of target DNA-engineered sequence specific DNA nuclease complexes, and cut the DNA at a pair of cleavage points that are within or near the binding sites. After the target DNA is cut, the DNA fragment between the two cutting sites is purified for further treatment and analysis, for example, for high throughput DNA sequencing. Because an engineered sequence specific DNA nuclease can be engineered to cut at any predetermined sequence, the DNA fragment of interest can be any size. In some preferred embodiments, the DNA fragment of interest cut and isolated using the engineered sequence specific DNA nucleases can be 5×10²-1×10⁸ base pairs long. In some more preferred embodiments, the DNA fragment of interest is 1×10⁴-1×10⁷ base pairs long. In some other embodiments, the DNA fragment of interest is 2×10⁴-5×10⁶ base pairs long.

In some embodiments of the invention, at least one of the engineered sequence specific DNA nucleases may continue to bind the DNA fragment of interest after the target DNA is cut. The engineered sequence specific DNA nuclease or a component thereof may include an affinity tag. The affinity tag aids the DNA fragment of interest—engineered sequence specific DNA nuclease complex to be captured on solid support or otherwise aids the isolation of the DNA fragment of interest.

The method described herein may be applied to genomic DNA enrichment for sequencing only the DNA sequence of interest on a target DNA. For example, the target DNA can be an entire chromosome, or even the whole genome, but the DNA sequence of interest is only 5×10²-1×10⁸-base pairs long. It would be inefficient to sequence the entire chromosome or even the entire genome to obtain information for this relatively small region of DNA. The DNA fragment including this region of interest may be cut out and isolated using the current method.

The DNA fragment of interest may be designed to include the entire DNA sequence of interest and also additional extra DNA sequences at both sides of the DNA sequence of interest. Thus, a precise cleavage point is not required even though the DNA is cut with sequence specificity. The extra DNA sequence provides the flexibility so that the binding sites and cleavage points may be moved around the target DNA to optimize cleavage efficiency and specificity.

In another embodiment, multiple pairs of engineered sequence specific DNA nucleases are employed in one reaction. Thus, multiple DNA fragments covering multiple regions of DNA sequences of interest may be cut and isolated in one run. In some applications, if the DNA sequence of interest is located near the end of the target DNA, then only one engineered sequence specific DNA nuclease is required for cutting and isolating the DNA fragment.

In yet another embodiment, multiple pairs of engineered sequence specific DNA nucleases are employed in one reaction to cut out the same DNA sequence of interested from the same target DNA, but at different cutting points, resulting in multiple DNA fragments all including the same DNA sequence of interest. By carrying out such redundant cuts for the same DNA sequence of interest, the overall efficiency, i.e. percentage of target DNA cut, may be increased. By combining the forgoing two embodiments, multiple DNA fragments covering the same DNA sequence of interest, as well as multiple DNA sequences of interest, may be cut and isolated in one run.

### BRIEF DESCRIPTION OF THE DRAWINGS

FIG. 1 is a schematic diagram that shows the working principle of an engineered sequence specific DNA nuclease that includes RecA and Ref.

FIG. 2 is a schematic diagram that shows the working principle of an engineered sequence specific DNA nuclease that includes a Transcription Activator Like Effector Nuclease, also known as TALEN.

FIG. 3 is a schematic diagram that shows the working principle of an engineered sequence specific DNA nuclease that includes a sequence specific chemical nuclease.

FIG. 4 is polyacrylamide gel stained with SYBR-Gold visualized with UV light. The gel shows that a 1.7 Kb fragment has been cut and released from a M13 DNA fragment.

FIG. 5 is a schematic diagram that shows the work flow of a demonstration of 2.3 Kb dsDNA pull-down.

FIG. 6 is bar graph that shows the results of the 2.3 Kb dsDNA pull-down experiment.

### DETAILED DESCRIPTION OF THE INVENTION

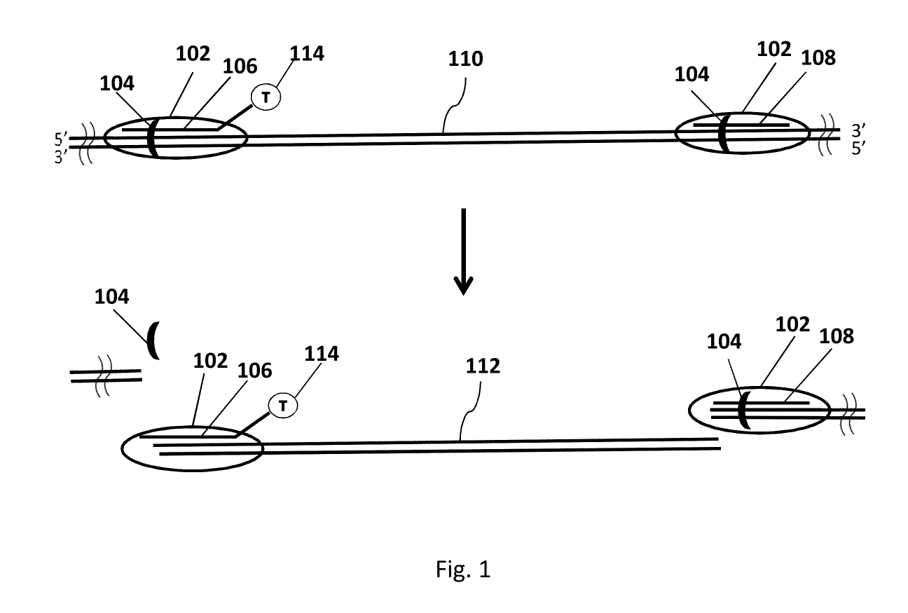

In an embodiment of the invention as shown in FIG. 1, the engineered sequence specific DNA nuclease is a composition that includes RecA 102 and Ref 104 protein and variants thereof, and a targeting oligonucleotide 106 and 108. Other ingredients of the scission reaction may include ATP and Mg²⁺. The use of RecA, Ref protein, and a targeting oligonucleotide as a sequence specific DNA nuclease has been described in U.S. patent application Ser. No. 13/208,985 by Cox et al. (the “Cox Application”), an article from the Cox Lab (Gruenig M. et al., J. of Biol. Chem. 286(10), 8240-8251, (2011), the “Cox Article”), and numerous other references. The Cox Application and Cox Article are incorporated herein by reference.

The RecA protein can be an E. Coli (strain K12) RecA protein (Uniprotsp POA7G6) or a mutant thereof as described the Cox Application. A RecA variant is defined broadly to include a RecA homolog derived from a common ancestor that performs the same function as RecA in other bacterial species or related families. Non-limiting examples of RecA homologs known in the art include RecA proteins from Deinococcus radiodurans, the RecA protein from Pseudomonas aeruginosa, and the RecA protein derived from Neisseria gonorrhoeae. A RecA variant is also defined to include a polypeptide having at least 40% sequence identity to E. Coli (strain K12) RecA protein and retains the RecA functionality. Preferably, the sequence identity is at least 90%, and more preferably, at least 98% sequence identity.

The Ref protein can be an Enterobacteria phage P1 Ref protein (Uniprotsp 35926) as described in the Cox Application. A Ref variant is defined broadly to include Ref homologs derived from common bacteriophage ancestors that perform the same function as Ref in other bacteriophage or bacterial species. Non-limiting examples of Ref homologs include the Enterobacteria phage φW39 recombination enhancement function (Ref) protein, the Enterobacteria phage P7 Ref protein, the recombination enhancement function (Ref) protein of Salmonella entrica subsp. Enterica serovar Newport str. SL317, and the putative phage recombination protein of Bordetella avium str. 197N. A Ref variant is also defined to include polypeptide variants having at least 75% sequence identity to the Enterobacteria phage P1 Ref protein (Uniprotsp 35926) and retains the Ref functionality. Preferably, the sequence identity is at least 90% to the reference sequence; more preferably, it is at least 98%.

Special RecA and Ref protein variants can be made to optimize the cutting efficiency, binding affinity before and after cutting, and/or sequence specificity. RecA-Ref fused protein variants can also be prepared through standard procedures, and screened for the desired properties.

The targeting oligonucleotide 106 and 108 can be a single-stranded DNA, RNA, LNA, PNA, or other DNA analogs. The other DNA analog may include phosphorothioate-DNA in which the phosphothiodiesters take place of the usual phosphodiesters, phosphorothioate-RNA, DNA in which thymidine is substituted with uridine, DNA in which guanidine is substituted with inosine. The DNA analog may include modified deoxyriboses, modified nucleobases, and modified phosphodiesters, which modifications may be currently known in the literature, for example, the DNA analogs described by Aboul-Fadl, Current Medicinal Chemistry, 12, 763-771 (2005), which is incorporated herein by reference, or later developed as long as the DNA analog is capable of sequence specific Watson-Crick base pairing with a complementary DNA.

The targeting oligonucleotide includes a targeting sequence that is 30-200 nucleotides long complementary to one of the strands on the intended biding site. Preferably, the targeting sequence is 50-150 nucleotides long. The entire targeting oligonucleotide may be 30-3000 nucleotides long.

The target DNA 110 is cleaved by incubating with the RecA, Ref, or variants thereof, the targeting oligonucleotide, ATP, and Mg²⁺ in a suitable buffer at a suitable temperature for a suitable length of time. The order of adding the foregoing reagents can be in any order. In a preferred embodiment, first the RecA and targeting oligonucleotide were incubated in a buffer with ATP, Mg²⁺, and an ATP regeneration system. The target DNA was added next, followed by the Ref. The solution was incubated at 37° C. for 3 hours before taken up for further treatment. Further details of the reaction condition for that containing a single targeting oligonucleotide can be found in the Cox application, the relevant part of which is incorporated here by reference.

After cleavage at both ends of the DNA fragment of interest 112, the DNA fragment can be separated by a number of methods. In some embodiments, the DNA fragment is separated based on the properties of the DNA fragment itself. The binding complex is broken up in denaturing conditions, or the proteins may be digested by proteases. The DNA fragment can then be separated by chromatographic method, gel electrophoresis, capillary electrophoresis, size exclusion filtration, or another method that separates DNA based on size and/or charge.

In another embodiment, the binding complex is likewise broken up, and the DNA fragment is captured on solid support that recognizes a sequence on the DNA fragment. The solid support may include a complementary single stranded DNA or DNA analog that is complementary to a sequence on one of the strands of the DNA fragment. The DNA fragment may be denatured to single strands to facilitate binding to the complementary single stranded DNA. In another variant, the solid support may include sequence recognizing proteins such as clusters of zinc finger proteins, or transcription activator like effectors that recognize a sequence on the DNA fragment. If the DNA fragment forms a secondary structure, the DNA fragment may be recognized and bound by a corresponding antibody or another protein, and be separated based on the antibody binding.

In some aspects of the embodiment, the DNA fragment may be separated based on the properties of the binding complex. In these cases, the binding complex is preserved after the target DNA is cleaved, and the DNA fragment is captured as a part of the binding complexes. The Cox article reported that the cleavage point by the Ref mediated cleavage is close to the 3′-end of the targeting oligonucleotide. It is expected that the binding complex formed between the RecA, Ref or their variants, targeting DNA, and the DNA fragment does not dissociate after the targeting DNA is cut. It is also expected that the other part of the target DNA on the other side of the cleavage point does dissociate from the binding complex because it does not have the complementary sequence of the targeting oligonucleotide.

In another aspect of the embodiment, the cleavage reaction is designed such that only one binding complex is expected to remain on the DNA fragment after the DNA fragment is cut out from the target DNA. In a configuration where the cutting out of a DNA fragment requires a pair of targeting oligonucleotides, the targeting oligonucleotides may be designed to bind the same strand in the target DNA. In another configuration where the DNA fragment is near the end of the targeting DNA, the targeting oligonucleotide is designed such that, when bound in a D-loop on the target DNA, the 5′-end of the targeting nucleotide is on the same side of the cleavage point as the DNA strand of interest.

An affinity tag 114 may be attached to the targeting oligonucleotide that remains bound to the DNA fragment. The affinity tag may be captured, causing the binding complex including the DNA fragment to be separated. The affinity tag may be biotin that can be recognized by avidin. The affinity tag may include multiple biotin residues for increased binding to multiple avidin molecules. The affinity tag may include a functional group such as an azido group or an acetylene group, which enables capture through copper(I) mediated click chemistry. Click chemistry is well known, and for example, is described in an article by Kolb and Sharpless at Drug Discovery Today, 8(24), 1128-1137 (2003). In some other variations, the affinity tag may include an antigen that may be captured by an antibody bound on a solid support. Other examples of affinity tag include, but not limited to, HIS-tag, Calmodulin-tag, CBP, CYD (covalent yet dissociable NorpD peptide), Strep II, FLAG-tag, HA-tag, Myc-tag, S-tag, SBP-tag, Softag-1, Softag-3, V5-tag, Xpress-tag, Isopeptag, SpyTag, B, HPC (heavy chain of protein C) peptide tags, GST, MBP, biotin carboxyl carrier protein, glutathione-S-transferase-tag, green fluorescent protein-tag, maltose binding protein-tag, Nus-tag, Strep-tag, and thioredoxin-tag.

In yet other variations, the targeting oligonucleotide is a solid support-bound oligonucleotide. In this case, the binding complex is formed on a solid support, the DNA scission process occurs on the solid support, and after scission, the binding complex including the DNA fragment remains bound on the solid support. The solid support may be glass, plastic, porcelain, resin, sepharose, silica, or other material. The solid support may be a plate that is substantially flat, gel, microbeads, magnetic beads, membrane, or other suitable shape and size. The microbeads may have diameter between 10 nm to several millimeters. The solid support may be non-porous or porous with various density and size of pores. With the DNA fragment captured on a solid support, unwanted DNA may be washed away. Then the DNA fragment can be released from the solid support, for example, through a denaturing process or by being cleaved from the solid support.

In another aspect of the embodiment, the cleavage reaction is designed such that both binding complexes remain bound on the DNA strand of interest after the target DNA is cut. This can be done where the cleavage of a DNA fragment requires a pair of targeting oligonucleotides, and the targeting oligonucleotides are designed to bind the opposite strands in the target DNA. The targeting oligonucleotides need to be oriented in such a way that the binding complexes are bound substantially inside the DNA strand of interest. Having two binding complexes on the DNA strand of interest makes it possible to design affinity tags on the RecA or Ref proteins or their variants, in addition to having design possibility of having affinity tag on the targeting oligonucleotide. Thus, one of more affinity tags can be put on the RecA, Ref, or variants thereof, and/or the targeting oligonucleotides. With both binding complexes bearing affinity tags, the capture of the DNA fragment after cutting may be enhanced.

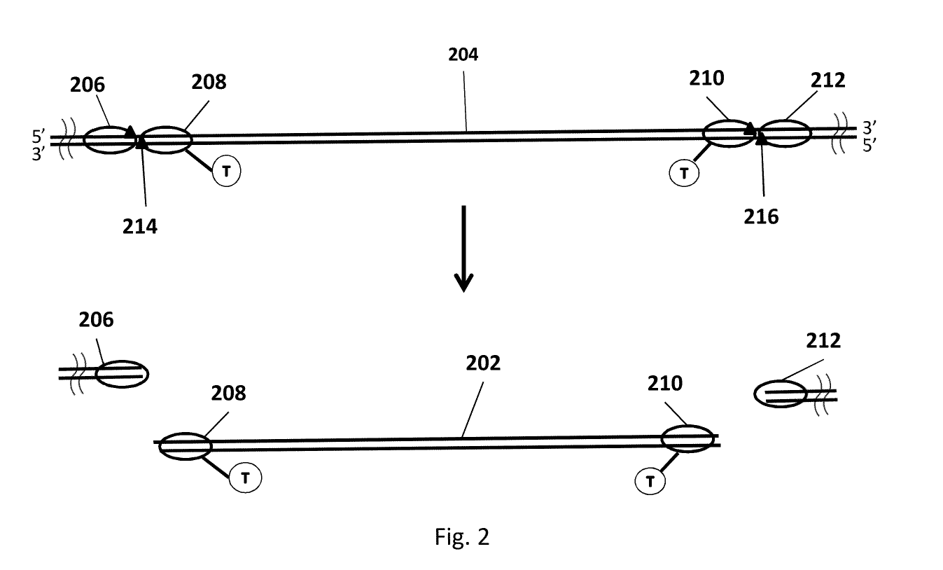

In another embodiment, the engineered sequence specific DNA nuclease is a Transcription Activator Like Effector Nuclease, also known as TALEN. TALEN can be constructed from Transcription Activator Like Effector, TALE, and a catalytic domain of nuclease. Numerous research articles and patents have described the preparation of TALEN and its use for efficient, programmable, and specific DNA cleavage. TALENs can be designed to recognize DNA sequences from 5 bp to 50 bp long, and theoretically any length practicably possible. For example, Miller et al. recently reported a method for generating such reagents based on TALE proteins from Xanthomonas that is linked to the catalytic domain of Fokl, and the use of these nucleases to achieve discrete edits or deletions on endogenous human NTF3 and CCR5 genes at efficiencies of up to 25%. Miller et al., Nature Biotechnology 29, 143-148 (2011). As shown in FIG. 2, to cut out a DNA fragment of interest 202 from a target DNA 204, four TALENs 206-212 maybe constructed. TALENs 206 and 208 will cleave the target DNA 204 at cleavage point 214, and TALENs 210 and 212 will cleave the target DNA at cleavage point 216. Each of the TALENs 206-212 can be designed to recognize DNA sequences of 5-50 bp long, preferably 12-25 bp long. In another embodiment, a TALEN is constructed from a TALE and a complete functional nuclease, and thus only one TALEN is required for carrying out a double stranded DNA scission. To cut out a DNA strand at two positions, only two TALENs are required. The TALEN can be engineered to stably bind on DNA after DNA scission.

Similar to the RecA/Ref embodiments, an affinity tag may be tethered to the TALENs 208 and 210 at a suitable site on the TALEN for easy isolation of the DNA fragment of interest. The affinity tag can be a biotin that can be bound by an avidin, an antigen that can be bound by an antibody, or another suitable moiety that can be bound with high affinity. The affinity tag can also be functional group that can be captured through a chemical reaction, for example, an azido group, an acetylene group or another group that can be captured through a click chemistry reaction. The solid support will include the respective counter parts for capturing the affinity tag. The solid support can be any shape, for example, plate and microbead.

In further embodiments, the TALEN is solid-supported. Thus, the TALEN mediated reaction may occur on a solid support, and the product DNA fragment of interest will remain on the solid support after DNA scission, facilitating isolation of the DNA fragment. In yet further embodiments, the product DNA can be separated from the TALEN after DNA scission, and the DNA fragment of interest can be isolated by characteristics of the DNA including size, charge, hydrophobicity, and/or sequence.

In yet another embodiment, the engineered sequence specific DNA nuclease is a sequence specific chemical nuclease, which includes a chemical nuclease linked to a sequence specific DNA binder. The sequence specific DNA binder can be an oligo, an engineered Zinc-finger protein, a TALE, or a DNA binding chemical substance such as distamycin. The oligo can be an oligodeoxyribonucleotide, oligoribonucleotide, or analogs thereof including phosphorothioate, zip nucleic acids, and other DNA or RNA analogs, for example, the DNA analogs described by Aboul-Fadl, Current Medicinal Chemistry, 12, 763-771 (2005), which is incorporated herein by reference. The chemical nuclease can be any chemical reagent that cleaves DNA, such as 1,10-phenanthroline-copper and derivatives thereof (Sigman et al. Chem. Rev. 93, 2295-2316, 1993; Chakravarty et al. Proc. Indian Acad. Sci. 114(4) 391-401, 2002), EDTA-Fe (Schultz and Dervan, J. Am. Chem. Soc. 105, 7748-7750, 1983). The double-stranded DNA cleaving activities of the chemical nucleases are well known in the literature, including the references cited above, which are incorporated herein by reference.

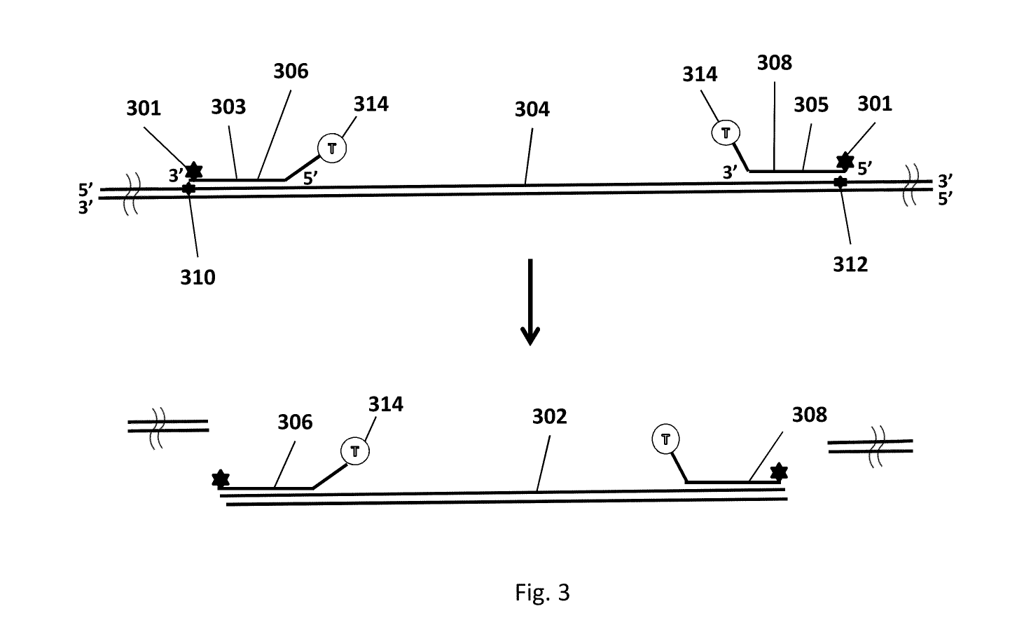

In an example as shown in FIG. 3, a sequence specific chemical nuclease 306 or 308 includes a chemical nuclease 301, a DNA 303 or 305, and an affinity tag 314. To cut out a DNA fragment of interest 302 from a target DNA 304, two sequence specific chemical nucleases 306 and 308 are required, which are designed to cut the target DNA at points 310 and 312. As shown, the sequence specific binder domains 303 and 305 of the sequence specific chemical nucleases 306 and 308 are designed to bind to the DNA strand of interest after the target DNA is cut at points 310 and 312. The sequence specific chemical nucleases 306 and 308 can be designed to have affinity tags 314 linked thereon. The affinity tags can be that described above for the TALEN embodiment. In another aspect, the sequence specific chemical nucleases can be solid support-bound in a way similar the above described embodiments.

### EXAMPLES

The following examples serve to demonstrate certain aspects of the present invention and do not limit it in any way.

### Example 1

### Demonstration of Cutting and Releasing 1.7 Kb Fragment by RecA Dependent Nuclease Activity of Ref

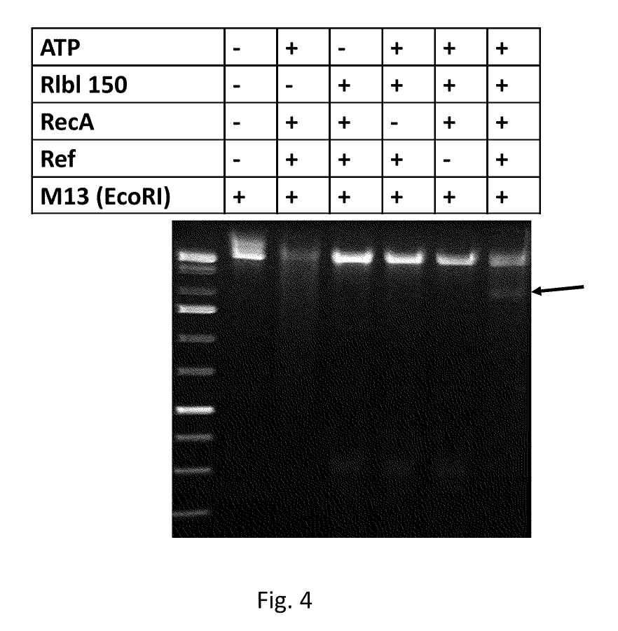

The reactions were carried out at 37° C. in RecA buffer (Tris-Acetate pH8, 60 mM magnesium, 10 units/ml pyruvate kinase and 3.5 mM phosphoenolpyruvate, 1 mM DTT) containing 10 U/mL pyruvate kinase and 3.5 mM phosphoenolpyruvate. Four Mnt of a 150 base target oligo (Rlb1 150) and 0.67 uM RecA(E38K) were incubated with above components for 10 minutes followed by the addition of 3 mM ATP and a 20 minute incubation. Eight micromolar nucleotides M13mp18 (linerized with EcoRI) were added followed by another 20 minutes incubation at 37° C. Then 48 nM Ref was added to the reaction. Three hours later, the reaction was treated with proteinase K (2 mg/ml) for 30 minutes at 37° C. The reaction was subjected to electrophoresis in 5% polyacrylamide gel with TBE buffer, stained with SYBR-Gold nucleic acid stain (Invitrogen) and visualized under UV light. As shown in FIG. 4, the 1.7 Kb fragment had been cut and released from other M13 DNA fragments (indicated by arrow).

### Example 2

### Demonstration of 2.3 Kb dsDNA Pull Down

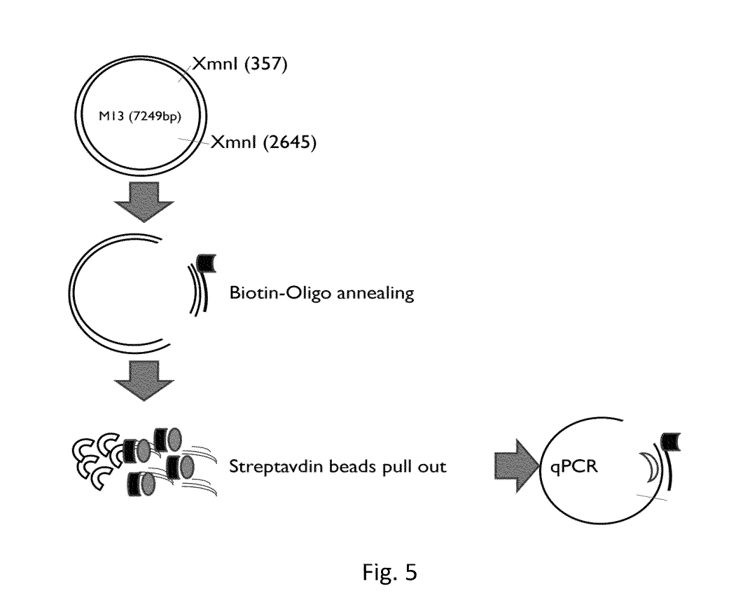

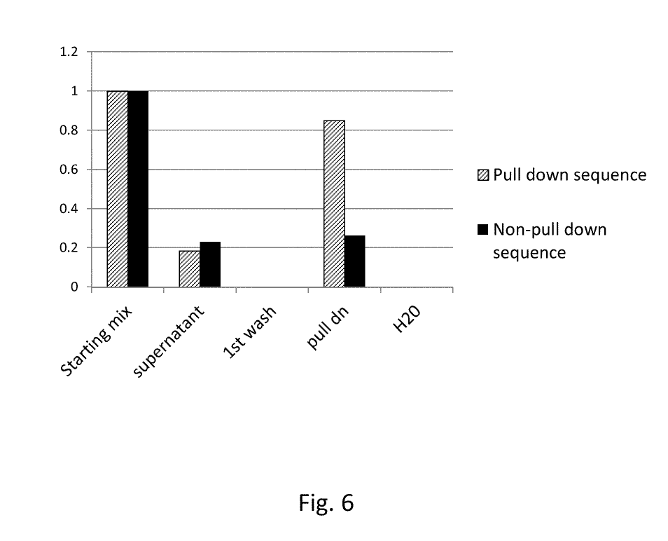

We tested the pull down efficiency of Streptavidin coated magnetic beads (Invitrogen) using M13 DNA (FIG. 5). The M13 DNA was digested with Xmn I (NEB) for two hours to generate two fragments 2.3 kb and 5 kb. The digested DNA was then annealed with a biotin labeled 99-nt long oligo that was complimentary to plus strand in the 2.3 kb fragment using a step cool-down procedure on a PCR machine. The mixture was then subjected to pull down assay following instructions provided by the manufacture. The final pull down product was treated for 3 min at 95° C. and pull down efficiency was detected by Taqman assay (FIG. 1). Sample aliquot from each step during the pull down process was used. The TaqMan assay results are shown in FIG. 6, with Ct value of each step being converted to percentage of total starting materials. Assay Probe III detected non-specific binding of DNA to magnetic beads. Probe II detected specific pull down of 2.3 Kb fragment. The results showed that about 80% of the 2.3 Kb fragment were pulled out from the digested M13 DNA mixes.

While embodiments and applications of this disclosure have been shown and described, it would be apparent to those skilled in the art that many more modifications and improvements than mentioned above are possible without departing from the inventive concepts herein. The disclosure, therefore, is not to be restricted except in the spirit of the appended claims.

What is claimed is:

1. A composition comprising an engineered sequence specific DNA nuclease, wherein:
said engineered sequence specific DNA nuclease is capable of cutting a target double stranded DNA with sequence specificity greater than eight base pairs long;
said engineered sequence specific DNA nuclease includes one or more affinity tags or is bound to a solid support; and
purification of a piece of DNA cut by said sequence specific DNA nuclease is facilitated by said affinity tag.

2. The composition of claim 1, wherein said engineered sequence specific DNA nuclease comprises:
a RecA protein or a variant thereof;
a Ref protein or a variant thereof; and
a targeting oligonucleotide;
wherein at least one of the above components includes an affinity tag or is bound to a solid support.

3. The composition of claim 2, comprising a RecA protein.

4. The composition of claim 2, comprising a Ref protein.

5. The composition of claim 2, comprising a targeting oligonucleotide that is a single-stranded DNA 30-1,000 nucleotides in length, wherein a 30-150 nucleotides long sequence is complementary to a sequence on one strand of the target double stranded DNA.

6. The composition of claim 2, comprising a RecA-Ref fused protein.

7. The composition of claim 2, wherein said affinity tag is selected from the group consisting of biotin, azido group, acetylene group, HIS-tag, Calmodulin-tag, CBP, CYD, Strep II, FLAG-tag, HA-tag, Myc-tag, S-tag, SBP-tag, Softag-1, Softag-3, V5-tag, Xpress-tag, Isopeptag, SpyTag, B, HPC peptide tags, GST, MBP, biotin carboxyl carrier protein, glutathione-S-transferase-tag, green fluorescent protein-tag, maltose binding protein-tag, Nus-tag, Strep-tag, and thioredoxin-tag.

8. The composition of claim 1, wherein said engineered sequence specific DNA nuclease comprises a transcription activator like effector nuclease.

9. The composition of claim 8, wherein said transcription activator like effector nuclease includes a subunit of a nuclease.

10. The composition of claim 8, wherein said transcription activator like effector nuclease includes a complete functional nuclease.

11. The composition of claim 8, wherein said affinity tag is selected from the group consisting of biotin, azido group, acetylene group, HIS-tag, Calmodulin-tag, CBP, CYD, Strep II, FLAG-tag, HA-tag, Myc-tag, S-tag, SBP-tag, Softag-1, Softag-3, V5-tag, Xpress-tag, Isopeptag, SpyTag, B, HPC peptide tags, GST, MBP, biotin carboxyl carrier protein, glutathione-S-transferase-tag, green fluorescent protein-tag, maltose binding protein-tag, Nus-tag, Strep-tag, and thioredoxin-tag.

12. The composition of claim 1, wherein said engineered sequence specific DNA nuclease comprises a sequence specific chemical nuclease that is a chemical nuclease linked to a sequence specific DNA binder.

13. The composition of claim 12, wherein said sequence specific DNA binder is selected from the group consisting of single stranded RNA or analog, single stranded DNA or analog, zinc-finger protein variant, transcription activator like effector, and distamycin.

14. The composition of claim 13, wherein said chemical nuclease includes one or more of the chemicals selected from the group consisting of phenanthroline or a derivative thereof, and EDTA or a derivative thereof.

15. The composition of claim 12, wherein said affinity tag is selected from the group consisting of biotin, azido group, acetylene group, HIS-tag, Calmodulin-tag, CBP, CYD, Strep II, FLAG-tag, HA-tag, Myc-tag, S-tag, SBP-tag, Softag-1, Softag-3, V5-tag, Xpress-tag, Isopeptag, SpyTag, B, HPC peptide tags, GST, MBP, biotin carboxyl carrier protein, glutathione-S-transferase-tag, green fluorescent protein-tag, maltose binding protein-tag, Nus-tag, Strep-tag, and thioredoxin-tag.

16. A method for cutting out a DNA fragment of interest from a target DNA, comprising:
contacting said target DNA with a composition of claim 2; and
isolating said DNA fragment of interest facilitated by said affinity tag.

17. The method of claim 16, wherein said DNA fragment of interest is 1×10⁴-1×10⁷ base pairs long.

18. The method of claim 16, wherein said composition includes at least one pair of targeting oligonucleotides, and the composition causes the target DNA to be cut by said engineered sequence specific DNA nuclease at both ends of the DNA fragment of interest in such a way that at least one pair of targeting oligonucleotides remain bound to the DNA fragment of interest after the target DNA is cut, and wherein one or more components of the engineered sequence specific DNA nuclease include said affinity tag or are bound to a solid support.

19. The method of 16, wherein said composition includes at least one pair of targeting oligonucleotides, and the composition causes the target DNA to be cut by said engineered sequence specific DNA nuclease at both ends of the DNA fragment of interest in such a way that only one targeting oligonucleotide remains bound to the DNA fragment of interest after the target DNA is cut, and wherein said targeting oligonucleotide that remains bound includes said affinity tag or is bound to said solid support.

20. The method of claim 16, wherein said composition includes one or more targeting oligonucleotides, and the composition causes the target DNA to be cut by said engineered sequence specific DNA nuclease at one end of the DNA fragment of interest in such a way that at least one of said targeting oligonucleotides remains bound to the DNA fragment of interest after the target DNA is cut, and wherein said one or more targeting oligonucleotides includes said affinity tag or is bound to said solid support.

21. A method for cutting out a DNA fragment of interest from a double stranded target DNA with sequence specificity of greater than eight base pairs long, comprising:
contacting said target DNA with one or more transcription activator like effector nuclease (“TALEN”), causing said targeting DNA to be cut at one or both ends of said DNA fragment of interest, wherein said TALEN includes an affinity tag or is bound to a solid support; and
isolating said DNA fragment of interest facilitated by said affinity tag or said solid support.

22. The method of claim 21, wherein said target DNA is cut at only one end of said DNA fragment of interest by one or more TALENs, and at least one of said TALENs remain bound to the DNA fragment of interest after the target DNA is cut, wherein said one or more TALENs include affinity tag or are bound to solid support.

23. The method of claim 21, wherein said target DNA is cut at both ends of said DNA fragment of interest by at least two TALENs, and at least two of said TALENs remain bound to the DNA fragment of interest after the target DNA is cut, wherein said TALENs includes affinity tag or are bound to solid support.

24. The method of claim 21, wherein said target DNA is cut at both ends of said DNA fragment of interest by at least two TALENs, and only one of said TALENs remains bound to the DNA fragment of interest after the target DNA is cut, wherein only said TALEN that remains bound includes said affinity tag or is bound to said solid support.

25. A method for cutting out a DNA fragment of interest from a double stranded target DNA with sequence specificity of greater than eight base pairs long, comprising:
contacting said target DNA with one or more sequence specific chemical nucleases, causing said targeting DNA to be cut at one or both ends of said DNA fragment of interest, wherein said one or more sequence specific chemical nucleases each includes an affinity tag or is bound to a solid support; and
isolating said DNA fragment of interest facilitated by said affinity tag or said solid support.

26. The method of claim 25, wherein said target DNA is cut at only one end of said DNA fragment of interest by one or more sequence specific chemical nucleases, and said sequence specific chemical nucleases remain bound to the DNA fragment of interest after the target DNA is cut.

27. The method of claim 25, wherein said target DNA is cut at both ends of said DNA fragment of interest by at least two sequence specific chemical nucleases, and at least two of said sequence specific chemical nucleases remain bound to the DNA fragment of interest after the target DNA is cut.

28. The method of claim 25, wherein said target DNA is cut at both ends of said DNA fragment of interest by two or more sequence specific chemical nucleases, and only one of said sequence specific chemical nucleases remains bound to the DNA fragment of interest after the target DNA is cut, only said sequence specific chemical nuclease that remains bound includes said affinity tag or is bound to said solid support.

29. A reagent kit comprising an engineered sequence specific DNA nuclease, wherein:
said engineered sequence specific DNA nuclease is capable of cutting a target double stranded DNA with sequence specificity greater than eight base pairs long;
said engineered sequence specific DNA nuclease includes one or more affinity tags or is bound to a solid support; and
purification of a piece of DNA cut by said sequence specific DNA nuclease is facilitated by said affinity tag.

30. The reagent kit of claim 29, wherein said engineered sequence specific DNA nuclease comprises following components, which may be mixed or separate:
a RecA protein or a variant thereof;
a Ref protein or a variant thereof; and
a targeting oligonucleotide;
wherein at least one of the above components includes an affinity tag or is bound to a solid support.

31. The reagent kit of claim 30, comprising a RecA protein.

32. The reagent kit of claim 30, comprising a Ref protein.

33. The reagent kit of claim 30, comprising a targeting oligonucleotide that is a single-stranded DNA 30-1,000 nucleotides in length, wherein a 30-150 nucleotides long sequence is complementary to a sequence on one strand of the target double stranded DNA.

34. The reagent kit of claim 30, wherein said affinity tag is selected from the group consisting of biotin, azido group, acetylene group, HIS-tag, Calmodulin-tag, CBP, CYD, Strep II, FLAG-tag, HA-tag, Myc-tag, S-tag, SBP-tag, Softag-1, Softag-3, V5-tag, Xpress-tag, Isopeptag, SpyTag, B, HPC peptide tags, GST, MBP, biotin carboxyl carrier protein, glutathione-S-transferase-tag, green fluorescent protein-tag, maltose binding protein-tag, Nus-tag, Strep-tag, and thioredoxin-tag.

35. The reagent kit of claim 30, comprising a RecA-Ref fused protein.

36. The reagent kit of claim 29, wherein:
said engineered sequence specific DNA nuclease comprises a transcription activator like effector nuclease; and
wherein said transcription activator like effector nuclease includes a subunit of a nuclease or a complete functional nuclease.

37. The reagent kit of claim 36, wherein said transcription activator like effector nuclease is bound to a solid support that is a plate, membrane, gel, magnetic bead, or microbead.

38. The reagent kit of claim 36, wherein said affinity tag is selected from the group consisting of biotin, azido group, acetylene group, HIS-tag, Calmodulin-tag, CBP, CYD, Strep II, FLAG-tag, HA-tag, Myc-tag, S-tag, SBP-tag, Softag-1, Softag-3, V5-tag, Xpress-tag, Isopeptag, SpyTag, B, HPC peptide tags, GST, MBP, biotin carboxyl carrier protein, glutathione-S-transferase-tag, green fluorescent protein-tag, maltose binding protein-tag, Nus-tag, Strep-tag, and thioredoxin-tag.

39. The reagent kit of claim 29, wherein said engineered sequence specific DNA nuclease comprises a sequence specific chemical nuclease that is a chemical nuclease linked to a sequence specific DNA binder.

40. The reagent kit of claim 39, wherein said sequence specific DNA binder is selected from the group consisting of single stranded RNA or RNA analog, single stranded DNA or analog thereof, zinc-finger protein variant, transcription activator like effector and distamycin.

41. The reagent kit of claim 39, wherein said chemical nuclease includes one or more of the chemicals selected from the group consisting of phenanthroline or a derivative thereof, and EDTA or a derivative thereof.

42. The reagent kit of claim 41, wherein said affinity tag is selected from the group consisting of biotin, azido group, acetylene group, HIS-tag, Calmodulin-tag, CBP, CYD, Strep II, FLAG-tag, HA-tag, Myc-tag, S-tag, SBP-tag, Softag-1, Softag-3, V5-tag, Xpress-tag, Isopeptag, SpyTag, B, HPC peptide tags, GST, MBP, biotin carboxyl carrier protein, glutathione-S-transferase-tag, green fluorescent protein-tag, maltose binding protein-tag, Nus-tag, Strep-tag, and thioredoxin-tag.
